(19) United States
(12) Patent Application Publication
(10) Pub. No.: US 2026/0283849 A1
(43) Pub. Date: Sep. 24, 2026

(54) OPHTHALMIC SURGICAL DEVICE AND METHOD OF USE FOR DELIVERY OF FLUIDS OR OTHER MATERIALS TO OCULAR TISSUES

(71) Applicant: NOVA EYE, INC., Fremont (CA)

(72) Inventors: Tony PUGATSCHEW, Kent Town (AU); Nick PREVIN, Kent Town (AU); Victor PREVIN, Kent Town (AU)

(73) Assignee: NOVA EYE, INC., Fremont (CA)

(21) Appl. No.: 19/693,511

(22) Filed: May 31, 2026

Related U.S. Application Data
(63) Continuation-in-part of application No. 19/153,332, filed on Jul. 26, 2023.

(30) Foreign Application Priority Data
Jul. 27, 2022 (AU) 2022902101
Dec. 5, 2022 (AU) 2022903707

(51) Int. Cl. A61F 9/007; A61B 17/00; A61B 17/04; A61B 90/30; A61M 25/00

(52) U.S. Cl. CPC A61F 9/00781; A61B 90/30; A61B 2017/00539; A61B 17/0469; A61M 2025/0042

(57) ABSTRACT

The invention relates to an ophthalmic surgical system for glaucoma surgery, comprising a handheld surgical device. The device includes a handheld housing and a microcatheter extendable from the handheld housing. The microcatheter comprises at least one tubular communicating element defining a lumen for fluid delivery and optionally communicating elements configured for optical transmission or sensing. An actuator is disposed on or carried by the handheld housing and a displacement mechanism is operatively coupled to the actuator and configured to advance and retract the microcatheter relative to the handheld housing. A light source can be coupled to or contained within the handheld housing, the light source being configured to illuminate at least a distal portion of the microcatheter. A fluid infusion system is coupled to the handheld housing or carried by the handheld assembly and configured to infuse fluid through the microcatheter. Each of the light source and the fluid infusion subsystem is independently disposed of within the handheld surgical device, carried by the handheld surgical device, or provided in an external unit coupled to the handheld surgical device.

### CROSS-REFERENCE TO RELATED APPLICATIONS

This application is a continuation-in-part of U.S. application Ser. No. 19/153,332 (MECHANICALLY AIDED FLUID DISPENSING DEVICE) being national phase of International Application No. PCT/AU2023/050681, filed July 26, 2023, and published as WO 20 24/020 632A1, which claims priority to Australian Provisional Patent Application No. 20 22 902101, filed July 27, 2022, and Australian Provisional Patent Application No. 20 22 903707, filed December 27, 2022. The entire contents of each of the foregoing applications are incorporated herein by reference.

### FIELD OF THE DISCLOSURE

The present disclosure relates generally to ophthalmic surgical devices, systems, and methods for delivering fluids or other materials to ocular tissues. More particularly, the disclosure relates to mechanically aided, handheld fluid dispensing devices for ophthalmic use, including devices configured for controlled microcatheter advancement, illumination, and fluid infusion during glaucoma surgery or related ocular procedures. Certain embodiments relate to devices and methods for canaloplasty, viscodilation, channelography, angiography, diagnostic or therapeutic agent delivery, and implant delivery within Schlemm's canal or other ocular outflow pathways. This is however not an exhaustive list.

### BACKGROUND

Glaucoma and related ocular conditions may be associated with increased intraocular pressure, impaired aqueous humor outflow, altered outflow resistance, collector channel dysfunction, tissue herniation, collapse or stenosis of Schlemm's canal, trabecular meshwork dysfunction, and other abnormalities affecting the eye's drainage pathways. Surgical treatment may include procedures directed to the trabecular meshwork, Schlemm's canal, collector channels, aqueous veins, distal outflow structures, the suprachoroidal space, the subconjunctival space, or other ocular tissues and anatomical pathways.

Catheter-based glaucoma procedures may involve introducing a microcatheter into an ocular outflow structure, advancing the microcatheter circumferentially or selectively, delivering fluid, viscodilating tissue, probing the patency of channels, identifying blockages or abnormal anatomy, delivering drugs or implants, or otherwise modifying ocular tissue or fluid flow.

Conventional systems may rely on separate devices or subsystems for illumination, catheter advancement, fluid delivery, imaging support, and control. Such systems may involve manual catheter advancement, external illumination, external pumps, external syringes, or multiple interconnected components that complicate use and reduce ergonomic simplicity. Some known devices may not provide adequate flexibility in terms of disposability, modularity, multifunctional catheter design, or tailored fluid-delivery behavior.

A need therefore exists for an ophthalmic surgical device that integrates or coordinates several functions that may include microcatheter advancement, handheld illumination, handheld fluid infusion, and optional diagnostic and therapeutic functions in a compact handheld architecture. A further need exists for a system that is broad enough to support multiple fluid types and delivery modes; multiple illumination modes and wavelengths; and the ability to perform both treatment and diagnostic assessment, including angiographic and channelographic assessment of ocular outflow anatomy.

### SUMMARY OF THE DISCLOSURE

In one aspect, the present disclosure provides an ophthalmic surgical device for glaucoma surgery including a handheld housing, a microcatheter extendable from the handheld housing, an actuator disposed on the handheld housing, and a displacement mechanism operatively coupled to the actuator and configured to advance and retract the microcatheter relative to the handheld housing. The device further includes a handheld light source configured to illuminate at least a distal portion of the microcatheter and a handheld fluid infusion system configured to infuse a fluid through the microcatheter.

In some embodiments, the microcatheter includes at least one flexible tubular communicating element and optionally at least one additional element selected from an optical element, a fluid delivery element, a reinforcing member, an electrical conductor, a sensor element, and combinations thereof.

In some embodiments, the microcatheter includes a single communicating element that performs multiple functions. The single communicating element may define a lumen for fluid delivery and may also transmit light through the wall of the catheter, through a coating, through the lumen, through fluid within the lumen, or through combinations thereof.

In some embodiments, the handheld light source is disposed within the handheld housing. In other embodiments, the handheld light source is outside the principal housing but remains mounted on, attached to, coupled to, or carried by the handheld device assembly such that the overall device remains handheld during use.

In some embodiments, the handheld fluid infusion system is disposed within the handheld housing. In other embodiments, the handheld fluid infusion system is outside the principal housing but remains mounted on, attached to, coupled to, or carried by the handheld device assembly such that the overall device remains handheld during use.

In some embodiments, the light source is configured to emit one or more colors or wavelengths, including red, green, blue, white, yellow, infrared, or combinations thereof. Different wavelengths may be selected to improve visualization, tissue contrast, optical penetration, compatibility with imaging, compatibility with dyes or contrast agents, or to account for photopic and other optical characteristics of ocular tissues.

In some embodiments, the fluid infusion system is configured for pressure-based delivery, displacement-based delivery, syringe-type volume exchange, pulsatile flow, continuous flow, bolus flow, metered delivery, rate-controlled delivery, or combinations thereof.

In some embodiments, the fluid infusion system is configured to deliver ophthalmic viscoelastic devices, saline, contrast, dye, drugs, biologics, irrigating fluids, diagnostic agents, therapeutic agents, or combinations thereof. In some embodiments, the microcatheter includes side ports, multiple lumens, or dual lumens to support selective or simultaneous delivery of different fluids.

In some embodiments, the device is configured for canal-based angiography, channelography, or related diagnostic procedures to identify occlusions, stenoses, herniations, abnormal flow regions, collector channel abnormalities, resistance changes, or treatment targets. In some embodiments, the device is configured to deliver or place an implant, optionally assisted by an ophthalmic viscoelastic device or other release mechanism.

In some embodiments, the implant comprises a suture element positioned at least partially within an internal passage of the microcatheter and releasably retained by engagement between at least one internal elongate member disposed within the microcatheter and the wall of the microcatheter. In some embodiments, there may be a first and second members. In some embodiments, the handheld fluid infusion system is configured to deliver a fluid, optionally an ophthalmic viscoelastic material, through the microcatheter with sufficient force to dislodge the suture element from engagement with the first and second internal elongate members and thereby advance at least a portion of the implant out of a distal end of the microcatheter for implantation in Schlemm's canal or another ocular target site.

In some embodiments, the fluid infusion system includes a motor or other drive source actuated by one or more user inputs, including a button on the device, another on-device actuator, voice control, a wired footswitch/remote, or a wireless footswitch/remote. Wireless communication may include Bluetooth, infrared, radio frequency, near-field communication, Wi-Fi, optical wireless communication, proprietary protocols, or combinations thereof.

In some embodiments, the light source, fluid infusion system, catheter, fluid chamber, and related components are fully disposable, fully reusable, or partially disposable and partially reusable. In some embodiments, the light source and/or fluid drive are reusable while the fluid chamber, sterile fluid path, and catheter are disposable.

### BRIEF DESCRIPTION OF THE DRAWINGS

FIG. 1 is a schematic sectional view of an ophthalmic surgical device including a handheld housing, a microcatheter, actuator, displacement mechanism, handheld light source, and handheld fluid infusion system according to one embodiment of the present disclosure.

FIG. 2 is a block diagram of some of the main components.

FIG. 3 is a schematic view of a single-communicating-element microcatheter configured to both transmit light and convey fluid.

FIG. 4 is a schematic view of a multi-element microcatheter including a communicating element and one or more additional elements.

FIG. 5 is a schematic view of a handheld device in which one or more functional subsystems are positioned outside a principal handpiece shell but remain attached to or carried by the handheld assembly.

FIG. 6 is a schematic view of different light transmission mechanisms through a microcatheter wall, coating, lumen, or fluid column.

FIG. 7 is a schematic view of dual-lumen and multi-lumen microcatheter embodiments.

FIG. 8 is a schematic cross-sectional view of a distal region of the microcatheter illustrating an implant comprising a suture element retained within an internal passage of the microcatheter between first and second internal elongate members.

FIGS. 9A-9C illustrate an optical sensor system including a broadband light source, an optical spectrum analyser, and an intrinsic or extrinsic optical sensor, wherein the optical signal may be modulated by changes in intensity, frequency, or phase, and wherein the sensor may be configured for single-point or multi-point sensing of one or more measured parameters.

FIG. 10 is a side view of a device with the internal configuration visible;

### LIST OF COMPONENTS

10—ophthalmic surgical device
12—handheld housing
14—microcatheter
16—actuator
18—displacement mechanism
20—handheld light source
22—handheld fluid infusion system
24—controller
26—motor
28—power source
30—fluid reservoir
32—Optical Fiber
34—Catheter Wall
36—Catheter Lumen
38—transmissive coating
40—outer jacket
42—reinforcing element
44—communicating element lumen
46—sensor element
48—electrical conductors
50—reinforcing member
52—dedicated lumen
54—suture implant
90—function connection hub
92—hollow inner section

### DETAILED DESCRIPTION OF THE DISCLOSURE

### General Ophthalmic Fluid Dispensing Platform

The embodiments described herein may be used with, incorporate, or further develop a mechanically aided fluid dispensing device for dispensing one or more fluids or substances into an eye or other ocular target region. In some embodiments, the device includes a body adapted to be held by a user, the body comprising a hollow inner section in which an inner body, catheter, carriage, reservoir, piston, or other movable member is located.

The movable member may be positioned in the hollow inner section and may be configured to protrude, translate, rotate, retract, advance, or otherwise move relative to the body. Movement of the movable member may cause fluid to be dispensed from a fluid chamber, storage chamber, cartridge, reservoir, syringe, tube, or other fluid source. In some embodiments, once the movable member is pushed or driven forward to a desired location, a dispensing unit is activated to move a defined amount of fluid from a fluid chamber to a needle, catheter, microcatheter, mixing section, delivery lumen, or other delivery path.

Movement of the movable member, catheter, microcatheter, inner body, or fluid displacement member may be aided by an external actuator, manual actuator, lever, tab, dial, slider, screw, pneumatic actuator, hydraulic actuator, gas cartridge, spring, motor, servo, stepper motor, linear motor, mechatronic drive, or other drive mechanism. The movement may be controlled manually, electronically, mechanically, pneumatically, hydraulically, or by combinations thereof.

In some embodiments, the device is adapted to dispense one or more fluids simultaneously or sequentially. The fluids may be stored in one or more internal or external fluid chambers. Different chambers may contain the same fluid or different fluids, including ophthalmic viscoelastic material, saline, balanced salt solution, dye, contrast agent, drug, biologic, therapeutic agent, diagnostic agent, flushing fluid, or combinations thereof.

The fluid chambers may be filled using a syringe, cartridge, prefilled module, external reservoir, or other source. In some embodiments, a chamber may be placed under vacuum, purged with fluid, sealed, or otherwise conditioned before use. An external chamber may be connected to the device using a fitting, thread, push lock, cam lock, luer, interference fit, flat-face fitting, barbed fitting, or other connection.

The dispensing force may be generated by gas pressure, pneumatic pressure, a piston, a screw, a press, a foot pedal, a lever, a spring, a motor, a pump, a pressurized cartridge, or other source. Gas or pressurized fluid may be controlled by a flow-limiting valve, pressure-reducing valve, check valve, regulator, on-off valve, solenoid valve, variable orifice, or other control arrangement.

In some embodiments, fluid delivery is displacement-based. For example, a screw, dial, piston, plunger, cam, diaphragm, bellows, or syringe-type component may move to displace a known amount of fluid. A gear train, planetary gearbox, pulley, rack, pinion, lead screw, or other transmission may establish a ratio between user input and fluid displacement or catheter movement.

The device may dispense multiple fluids at controlled ratios. In some embodiments, multiple fluid chambers feed a common delivery path, mixing region, tube, catheter, or microcatheter. Fluid mixing may occur in a static mixer, dynamic mixer, helical mixer, mesh, baffle, vane, bead arrangement, cross-sectional flow path, spiral path, or other mixing structure. Flow ratios may be set by orifice size, nozzle geometry, valve selection, actuator travel, pressure, displacement, or programmed control.

A catheter, needle, delivery catheter, fibre tube, hollow tube, flexible needle, hypodermic needle, or microcatheter may be connected to or extend from the device. The delivery element may include a hollow section for fluid delivery, an optical fibre or light-transmitting structure for illumination, one or more tubes, and one or more support or reinforcement structures. The delivery element may be configured for insertion into ocular tissue or an ocular pathway and may be extendable or retractable relative to the body.

The delivery element may have any suitable length, diameter, stiffness, tip geometry, port arrangement, material, coating, marker, or reinforcement structure. In some embodiments, a delivery catheter or microcatheter includes a hollow section for delivery of fluid and a light-transmitting element for illumination of a distal region or tip. In other embodiments, a single structure may participate in both fluid delivery and light transmission.

The device may include an illumination module, optical fibre, light source, laser diode, LED, visual indicator, fluorescence source, light guide, optical coupling, or other illumination system. The light source may be internal to the device, external to the device, or located in a module attached to or carried by the device. Illumination may be used to identify the device state, visualize a distal tip or catheter, track a delivered fluid, excite a fluorescent material, or assist a surgical procedure.

The light source may emit visible light, ultraviolet light, infrared light, or another wavelength, including red, green, blue, white, or combinations thereof. A duty cycle, intensity, wavelength, modulation, pulse pattern, or emission mode may be controlled according to procedure requirements, power use, visualization, sensor feedback, or safety limits.

The device may include one or more power sources, including an internal or external battery, rechargeable battery, button cell, coin cell, external power supply, wireless power arrangement, or other energy source. A power source may be positioned inside the device or in a module, handle portion, cartridge, rear extension, sidecar, or other attached component.

The device may include a user interface with one or more buttons, levers, sliders, dials, switches, triggers, displays, lights, speakers, buzzers, gauges, screens, indicators, or combinations thereof. A user interface may communicate device state, pressure, fluid volume, catheter position, battery state, priming state, occlusion state, proper cartridge loading, or readiness for use.

The device may include one or more sensors. Sensors may detect pressure, flow, displacement, force, motor current, catheter position, tissue resistance, optical return, fluorescence, temperature, cartridge identity, fluid volume, catheter loading, priming status, or other information. Sensor data may be displayed, stored, used for control, used to indicate occlusion or resistance, or used to regulate fluid delivery, catheter advancement, illumination, or implant release.

The device may include a communication module configured for wired or wireless communication, including Bluetooth, radio frequency, near-field communication, Wi-Fi, optical communication, infrared communication, or other communication. The device may receive control commands or transmit sensor, status, calibration, use, or procedure data.

The device may be programmed to undertake a fluid dispensing operation. A program may recognize that a fluid chamber, reservoir, cartridge, or other component has been placed into the device, prime the fluid path, confirm readiness, select a delivery volume, select a pressure profile, deliver pulses, deliver a bolus, deliver a set volume, deliver for a set duration, or coordinate catheter movement and fluid discharge.

The device may be fully disposable, fully reusable, or partially disposable and partially reusable. In some embodiments, a reusable handpiece includes a drive, power source, controller, communication hardware, or light source, while a disposable component includes a catheter, microcatheter, fluid chamber, sterile fluid path, reservoir, mixing section, implant carrier, or patient-contacting portion.

### Handheld Glaucoma Surgical Device

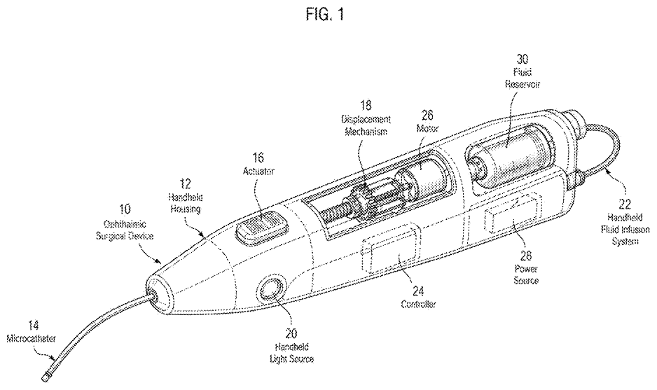

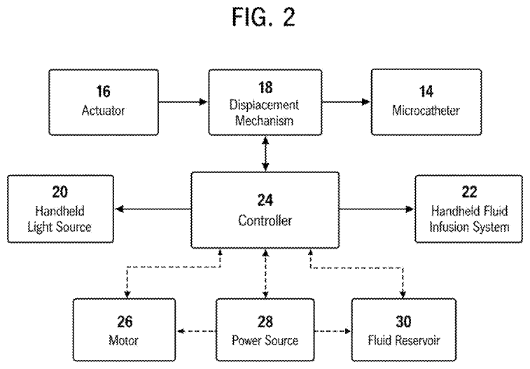

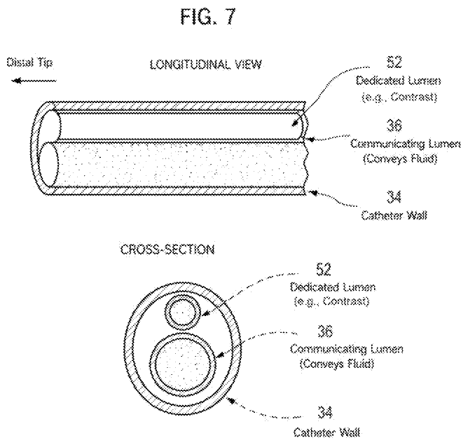

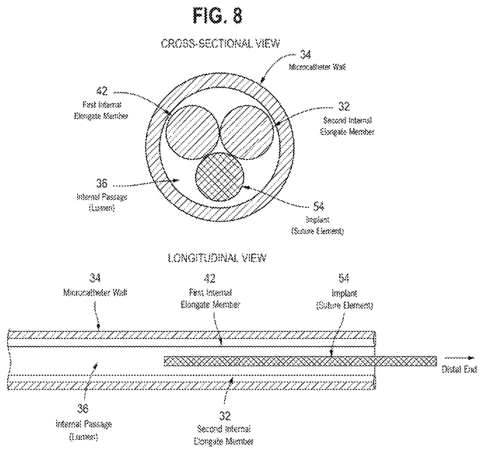

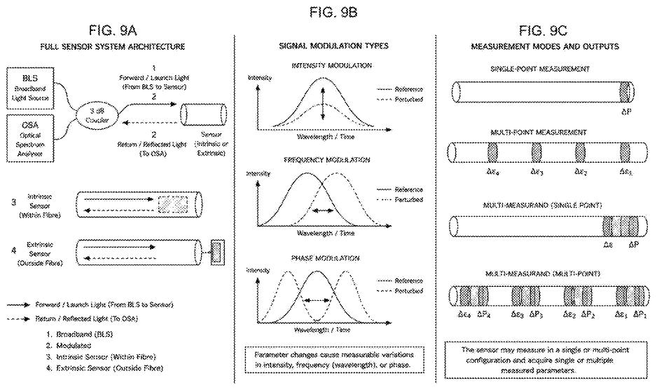

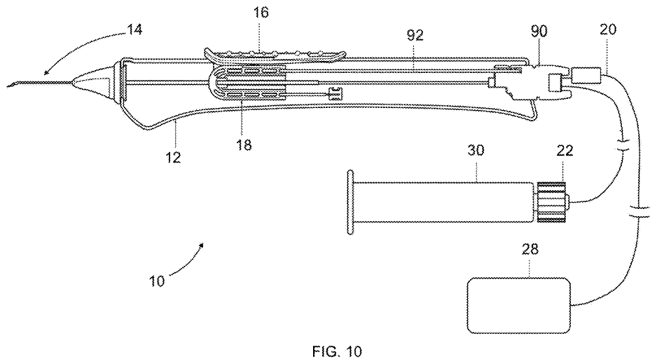

Referring to FIGS. 1-10, and according to various embodiments, an ophthalmic surgical device 10 includes a handheld housing 12, a microcatheter 14 extendable from the handheld housing 12, an actuator 16 disposed on the handheld housing 12, and a displacement mechanism 18 operatively coupled to the actuator 16 and configured to advance and retract the microcatheter 14 relative to the handheld housing 12.

The device 10 further includes a handheld light source 20 configured to illuminate at least a distal portion of the microcatheter 14 and a handheld fluid infusion system 22 configured to infuse a fluid through microcatheter 14.

The device 10 may further include one or more of a controller 24, a motor 26, a power source 28 and a fluid reservoir 30.

The device may be used in glaucoma surgery, including but not limited to canaloplasty, viscodilation, transluminal catheterization, trabeculotomy, goniotomy assisted transluminal trabeculotomy (GATT) procedures, treatment of Schlemm's canal, treatment of collector channels, distal outflow assessment, canal-based angiography, channelography, drug delivery, contrast delivery, implant delivery, and related therapeutic and diagnostic procedures.

As used herein, the term ‘handheld’ means that the corresponding structure or subsystem is configured to be at least primarily borne and manipulated by the user as part of the surgical instrument during normal use. A subsystem may remain handheld even if it is outside a principal handpiece shell, provided it is attached to, mounted on, coupled to, or carried by the overall device assembly such that the device remains operator-borne and manually manipulable as a unit. Footswitches, or other remote actuation devices do not limit the ability of the device to be considered handheld.

In a preferred embodiment, the ophthalmic surgical device 10 comprises a handheld housing 12 configured for single-handed use in glaucoma surgery, a microcatheter 14 extendable from the housing 12, an actuator 16 positioned on the housing 12 for thumb or finger operation, and a displacement mechanism 18 coupled to the actuator 16 to provide controlled advancement and retraction of the microcatheter 14 relative to the housing 12. The microcatheter 14 preferably comprises a flexible tubular communicating element configured to deliver fluid into Schlemm's canal and further comprises an optical element 32 or light-transmitting structure enabling illumination of at least a distal portion of the microcatheter 14. A handheld light source 20, preferably an LED or laser-based source, is optically coupled to the microcatheter 14 and is configured to emit visible light, preferably red, green, white, or combinations thereof, to facilitate intraoperative visualization of catheter position within ocular anatomy. The device 10 further preferably includes a handheld fluid infusion system 22 configured to deliver an ophthalmic viscoelastic device through the microcatheter 14 under controlled pressure or displacement-based delivery, optionally in a pulsatile manner.

In a particularly preferred form, the infused fluid is a shear-thinning viscoelastic material comprising hyaluronic acid or sodium hyaluronate, and the fluid path includes one or more narrowed or non-uniform regions configured to increase shear during delivery and thereby reduce apparent viscosity during infusion. In some preferred embodiments, the microcatheter 14 includes dual lumens to permit selective delivery of viscoelastic, contrast agent, or drugs. The light source 20 and fluid infusion system 22 may each be housed within the handheld housing 12 or mounted externally thereto while remaining part of a handheld assembly.

In some preferred embodiments, reusable drive, power, and illumination components are combined with a disposable fluid chamber, sterile fluid path, and microcatheter 14. The preferred embodiment may further be used for canal-based channelography or angiography to identify occlusions, herniations, and outflow abnormalities, and may additionally be used to deliver an implant, optionally with viscoelastic-assisted release.

The handheld housing 12 may take any suitable shape, including elongate, pen-like, curved, pistol-grip, ergonomic contoured, or hybrid forms. The housing 12 may include one or more finger grips, thumb rests, palm supports, texture features, anti-slip features, sterile drape-compatible regions, control regions, or attachment interfaces.

The particular housing geometry may be selected according to the surgical approach, hand position, required stability, and extent of integrated functionality. For example, an elongate or pen-like housing may be preferred where fine rotational control is desired. A curved or contoured housing may be preferred where the catheter entry angle, surgeon wrist posture, or clearance around a gonioscopic lens favors an offset grip. A pistol-grip or hybrid housing may be preferred where greater actuation force, separation of advancement and infusion controls, or a larger internal drive or reservoir is desired. Texture, grip, and control-region placement may be chosen to reduce slippage, distinguish controls by touch, and maintain stable catheter position during fluid delivery or implant release.

The housing 12 may be formed from one or more polymers, elastomers, metals, ceramics, composite materials, or combinations thereof. The housing 12 may be rigid, semi-rigid, or include flexible portions. The housing 12 may be single-use, reusable, or partly reusable and partly disposable.

Material selection for the housing may depend on whether the housing is intended to be disposable, reusable, sterilizable, low cost, lightweight, rigid, or compatible with electronic and optical subsystems. Polymers may be preferred for single-use or molded sterile components because they can reduce cost and weight and can support complex internal features. Metals or reinforced composites may be preferred for reusable drive modules, precision alignment structures, or regions subject to higher mechanical loads. Elastomeric portions may be preferred at grip regions, seals, or interfaces where comfort, tactile feedback, fluid isolation, or tolerance absorption is desired.

The housing 12 may contain some or all of the following: a microcatheter path, an actuator 16, a displacement mechanism 18, a light source 20, a fluid chamber, a fluid drive, a battery, control electronics, sensors, a communication module, a speaker or microphone for voice input, a wireless module, or a detachable interface for disposable components.

The choice of which subsystems are contained within the housing may reflect a balance between compactness, sterility, ergonomics, serviceability, and disposability. Integrating the catheter fluid path, drive, light source, fluid chamber, and power source into the housing may reduce external connections and simplify setup. Separating one or more subsystems may be preferred where a larger reservoir, higher-capacity battery, reusable electronics, improved thermal management, easier sterilization, or lower disposable cost is desired. A modular architecture may also permit different procedure-specific cartridges to be used with a common reusable handpiece.

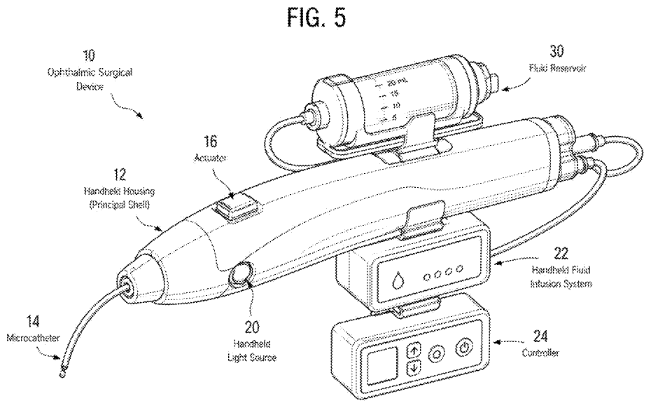

In some embodiments, one or more functional components are not within the principal housing shell but are instead in a sidecar module, rear extension, clip-on assembly, cartridge, handle-adjacent module, or other structure attached to or carried by the handheld assembly, as schematically illustrated in FIG. 5.

Locating a subsystem in a sidecar, rear extension, clip-on assembly, cartridge, or handle-adjacent module may be useful when the subsystem would otherwise increase the handpiece diameter or interfere with a preferred grip. Rear-mounted modules may shift mass proximally and improve balance. Side-mounted modules may facilitate visual inspection or cartridge replacement. Clip-on or detachable modules may allow a common instrument body to support different light, reservoir, sensor, or implant-delivery configurations without redesigning the principal housing.

### Microcatheter, Actuation, and Illumination Arrangements

The microcatheter 14 is extendable from the handheld housing 12 and is configured to be advanced and retracted relative to the handheld housing 12. In some embodiments, the microcatheter 14 is configured to be introduced into Schlemm's canal. In other embodiments, the microcatheter 14 may be directed into collector channels, aqueous veins, the suprachoroidal space, subconjunctival pathways, tissue planes, or other ocular anatomy.

The microcatheter 14 may have any suitable dimensions for ocular procedures. It may include an atraumatic distal tip, a rounded distal region, a tapered distal region, a closed distal tip, an open distal tip, side ports, distal ports, lateral ports, multiple ports, or combinations thereof.

Microcatheter dimensions and distal geometry may be selected according to the target anatomy and the desired balance between pushability, trackability, flow capacity, and atraumatic navigation. A smaller and more flexible distal region may be preferred for circumferential travel in Schlemm's canal or for entry into delicate collector-channel regions. A larger lumen or reinforced proximal region may be preferred where higher flow, implant loading, improved torque response, or resistance to buckling is desired. An open distal tip may be preferred for axial fluid delivery or implant release. This can be coupled with a rounded distal tip with may be preferred where atraumatic advancement is prioritized.

The microcatheter 14 comprises at least one flexible tubular communicating element. The communicating element may define one or more lumens or pathways for fluid, optical energy, electrical communication, sensor signals, guide elements, or other functions. In some embodiments, the microcatheter 14 optionally includes one or more additional elements selected from an optical element, a fluid delivery element, a reinforcing member, an electrical conductor, a sensor element, or combinations thereof.

A single communicating element may be selected where reduced catheter diameter, simplified manufacture, and fewer interfaces are important. Additional elements may be selected where a single element cannot provide the desired combination of optical performance, fluid capacity, mechanical support, sensing, or electrical communication. For example, a dedicated optical fiber may improve light transmission efficiency, a reinforcing member may improve pushability or kink resistance, and a sensor element may provide feedback useful for pressure monitoring, tissue interaction assessment, or confirmation of implant release.

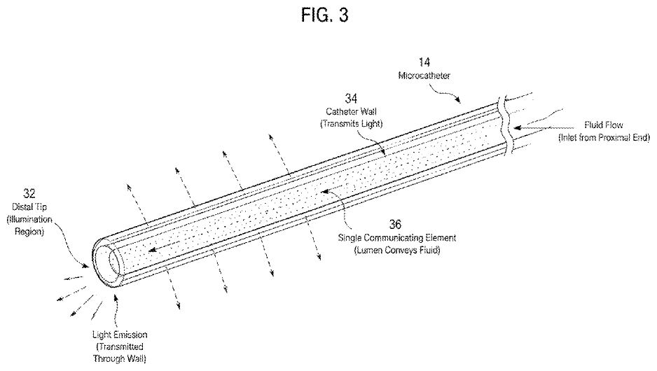

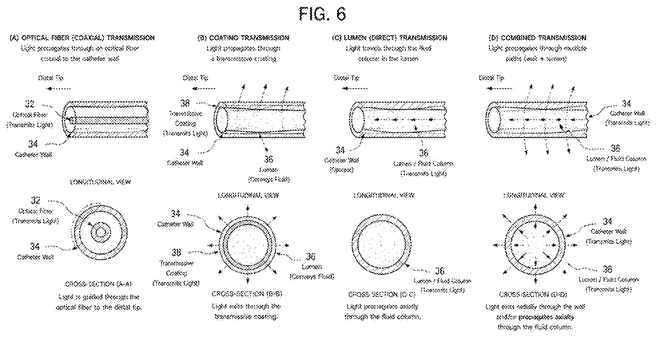

In some embodiments, the microcatheter 14 consists essentially of a single flexible tubular communicating element that performs multiple functions. For example, a single catheter tube may convey fluid, transmit light, provide structural support, and serve as the primary navigating element, as generally shown in FIGS. 3 and 6.

In some embodiments, the wall of the catheter transmits light. The wall may be transparent, translucent, semi-transmissive, or optically engineered. The wall may guide light longitudinally, radially, circumferentially, or in a mixed manner.

Wall-based light transmission may be useful because it avoids dedicating a separate lumen or fiber to illumination and may preserve cross-sectional areas for fluid delivery or implant accommodation. This approach may be preferred for simplified single-element catheters or where diffuse visualization along the distal catheter is beneficial. A dedicated optical element may instead be preferred where higher brightness, defined emission location, reduced optical loss, or more controlled coupling to imaging systems is desired.

In some embodiments, a coating on or within the catheter facilitates light transmission, reflection, refraction, redirection, diffusion, emission, or confinement. The coating may be reflective, partially reflective, diffusive, fluorescent, phosphorescent, luminescent, refractive, index-shifting, index-matching, scattering, or otherwise optically functional.

An optically functional coating may be selected to tailor how light exits or travels along the catheter without materially changing the main catheter structure. A diffusive coating may be preferred where broad visibility of the catheter path is desired. A reflective or partially reflective coating may be preferred to confine light, increase distal brightness, or reduce glare. A fluorescent or luminescent coating may be preferred where excitation and emission wavelengths are chosen to improve contrast against ocular tissue or to support camera-based detection.

In some embodiments, light is transmitted through the lumen. A fluid present in the lumen may serve as a refractive index medium, optical coupling medium, waveguiding medium, or light-propagation medium. In some embodiments, the fluid itself may be selected or conditioned for optical transmission, for example by refractive index, clarity, dye content, fluorescence, or optical density.

Lumen-based optical transmission may be advantageous where the lumen is already present for fluid delivery and where the fluid can act as an optical coupling or waveguiding medium. This arrangement may reduce the need for a separate optical fiber and may permit fluid selection to influence visibility. However, a lumen-based optical path may be less preferred where debris, dye concentration, implant components, or variable fluid optical properties could impair consistent illumination.

In some embodiments, the single communicating element transmits light through one or more of the wall, coating, lumen, fluid column, or combinations thereof.

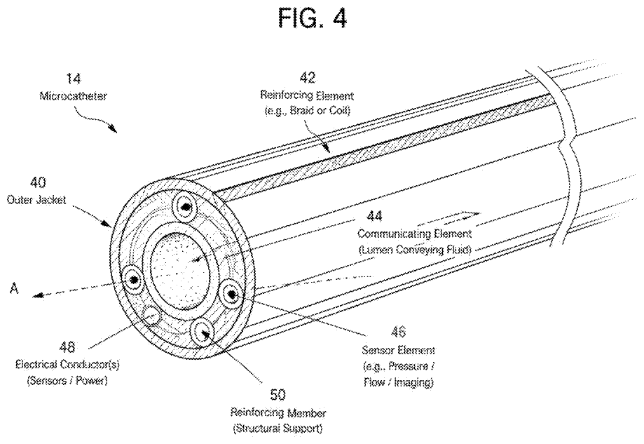

In some embodiments, the microcatheter 14 includes a tubular communicating element together with one or more additional elements. The additional elements may include an optical element, such as an optical fiber, waveguide, or light-transmitting member; a fluid delivery element, such as a second lumen or supplemental tube; a reinforcing member, such as a coil, braid, ribbon, filament, hypotube segment, spring element, or superelastic member; an electrical conductor; or a sensor element, as schematically shown in FIG. 4.

The additional elements may be arranged concentrically, in parallel, helically, multilayered, embedded within the wall, within the lumen, adjacent the lumen, or otherwise integrated with the catheter.

The arrangement of additional elements may be chosen to control catheter profile, stiffness distribution, optical coupling, lumen area, and manufacturing complexity. A concentric arrangement may provide uniform bending behavior and a smooth outer profile. Parallel or embedded arrangements may be preferred when preserving a larger central lumen or separating fluid, optical, and sensing functions is desirable. Helical or multilayered arrangements may be used to improve flexibility, torque response, kink resistance, or circumferential light emission while maintaining a small outer diameter.

The microcatheter 14 may include a single lumen, dual lumens, multiple lumens, and/or one or more side ports. A first lumen may be used for one fluid and a second lumen for another fluid. A first lumen may be used for an ophthalmic viscoelastic device and a second lumen for contrast, dye, saline, a drug, or another agent. In some embodiments, one lumen may also support sensing, aspiration, optical coupling, or pressure monitoring.

Side ports may be provided near the distal tip, proximal to the distal tip, distributed longitudinally, circumferentially. Side ports may be used to direct flow laterally into collector channels or at the trabecular meshwork. Port size, number, spacing, orientation, and geometry may be selected to influence delivery pressure, distribution, penetration, and directionality.

The microcatheter 14 may be formed from polymers, elastomers, silicones, polyurethanes, polyamides, acrylics, PEBAX-type materials, polyethylene, nylon materials, polyester materials, and combinations thereof.

Catheter material selection may depend on flexibility, optical transmission, biocompatibility, bonding compatibility, coefficient of friction, kink resistance, and ability to tolerate sterilization. Softer elastomeric or polyurethane materials may be preferred for atraumatic distal navigation. Polyamide, PEBAX-type, or reinforced polymer regions may be preferred where pushability, dimensional stability, or thin-wall strength is desired. Materials with suitable optical clarity or refractive properties may be selected where the catheter wall or lumen participates in light transmission.

The microcatheter 14 may include radiopaque materials, optical markers, lubricious coatings, hydrophilic coatings, hydrophobic coatings, anti-kink structures, variable stiffness regions, tapered reinforcement, or distal atraumatic features. In some embodiments, proximal regions are relatively stiffer for pushability and distal regions are relatively more flexible for atraumatic navigation. Reinforcement may be provided by coils, braids, filaments, ribbons, wires, superelastic members, spring structures, or combinations thereof.

Markers, coatings, reinforcement, and variable-stiffness features may be selected according to the feedback modality and the mechanical demands of the procedure. Lubricious or hydrophilic coatings may be preferred to reduce friction during canal traversal. Radiopaque or optical markers may be preferred where confirmation under imaging or direct visualization is desired. Anti-kink structures and tapered reinforcement may be preferred where a long catheter length must be advanced through a compact handheld device without buckling while still preserving a soft distal segment for tissue protection.

The actuator 16 is disposed on the handheld housing 12 and may be manually operated by the user. The actuator 16 may be an actuator, slider, trigger, button, rocker, wheel, thumbwheel, lever, touch-sensitive region, capacitive surface, pressure-sensitive region, switch, or combinations thereof.

The actuator form may be selected according to the force required, desired travel resolution, surgeon hand position, and need to avoid unintended activation. A slider or thumbwheel may be preferred for gradual catheter advancement and tactile feedback. A trigger or lever may be preferred where larger travel or greater mechanical advantage is required. A button or rocker may be preferred for electronic control, indexed advancement, fluid bolus delivery, or mode selection. Touch-sensitive or capacitive controls may reduce moving parts, but mechanical controls may be preferred where glove compatibility, tactile confirmation, and reliability in a wet surgical field are priorities.

The actuator 16 may directly mechanically control the displacement mechanism 18 or may provide an electrical, electronic, optical, or other control signal to a control system that drives the displacement mechanism 18. The actuator 16 may support continuous movement, indexed movement, stepped movement, coarse and fine modes, locking, or programmable behavior.

The displacement mechanism 18 is operatively coupled to the actuator 16 and is configured to advance and retract the microcatheter 14 relative to the handheld housing 12. The displacement mechanism 18 may include wheels, rollers, belts, pulleys, clamps, lead screws, racks, pinions, gears, sliding supports, or combinations thereof.

In some embodiments, the displacement mechanism 18 includes a guide sleeve or support path to reduce buckling or kinking within the device. In some embodiments, the displacement mechanism 18 is configured to provide precise advancement suitable for delicate ocular navigation.

In some embodiments, a transmission arrangement is present between the actuator 16 and the displacement mechanism 18. The transmission may include a gear train, pulley arrangement, belt, cable, lead screw, or other ratio-establishing arrangement. The selected ratio may increase precision, reduce sensitivity, increase output force, or provide coarse and fine advancement modes. In some embodiments, a pulley-based ratio such as about 2:1 may be used, though other ratios may be used.

In some embodiments, a lower transmission ratio is selected where finer microcatheter positioning is desired, for example while navigating Schlemm's canal or positioning for implant release, because reduced output travel per unit user input may improve tactile control and reduce overshoot. In other embodiments, a higher transmission ratio may be preferred because it may reduce the number of user inputs needed to move the microcatheter through a greater distance. This may allow the user to complete a circumferential navigation of Schlemm's canal with a single finger stroke.

The device 10 includes a handheld light source 20 configured to illuminate at least a distal portion of the microcatheter 14. In some embodiments, the light source 20 is within the handheld housing 12. In other embodiments, the light source 20 is outside the main housing shell but remains attached to, mounted on, coupled to, or carried by the handheld assembly.

The light source 20 may be in the main handpiece, a side-mounted module, a rear-mounted module, a detachable cartridge, a clip-on component, a separate light module physically borne by the handpiece, or another handheld arrangement.

The light source 20 may include one or more LEDs, laser diodes, VCSELs, edge emitters, electroluminescent sources, phosphor-converted emitters, fiber-coupled emitters, and other light-emitting devices. Light may be continuous, pulsed, modulated, selectable in intensity, selectable in wavelength, or spectrally tunable.

The light-emitting technology may be selected according to brightness, heat generation, spectral width, coupling efficiency, cost, and safety. LEDs may be preferred for compactness, low cost, broad wavelength availability, and relatively simple thermal management. Laser diodes or VCSELs may be preferred where high coupling efficiency into a fiber or narrow spectral emission is desired. Pulsed or modulated light may be preferred to reduce average heat, improve detection by imaging systems, encode information, or distinguish device illumination from ambient surgical illumination.

The light source 20 may emit one or more colors or wavelengths, including red, green, blue, white, amber, yellow, cyan, magenta, infrared, near-infrared, ultraviolet, or combinations thereof.

Different wavelengths may be selected for different purposes. In some embodiments, wavelength selection enhances visual contrast between the microcatheter 14 and ocular tissue. In some embodiments, wavelength selection is based on photopic optical response, tissue absorption, tissue scattering, transmission characteristics, interaction with blood or pigments, or visibility against the tissue background of the eye.

In some embodiments, green light may enhance visibility against reddish tissue backgrounds; red light or longer wavelengths may provide different tissue penetration or visibility characteristics; blue light or shorter wavelengths may enhance contrast, coating visibility, dye interaction, or surface detail; and white light may provide general-purpose visualization.

In some embodiments, multiple colors may be emitted sequentially, simultaneously, or in a programmed pattern. In some embodiments, color may encode device state, lumen selection, fluid type, pressure state, treatment mode, or diagnostic mode. In some embodiments, color selection may support direct visualization, machine vision, fluorescence-based assessment, channelography, angiography, or image enhancement.

Where an optical element is present, the light source 20 may be optically coupled to the optical element. Where no dedicated optical element is present, the light source 20 may be coupled to the wall, coating, lumen, or fluid column of the microcatheter 14. Light may be coupled through connectors, ferrules, interfaces, lenses, index-matching media, waveguides, or direct coupling arrangements.

### Fluid Infusion and Diagnostic Delivery

The device 10 further includes a handheld fluid infusion system 22 configured to infuse a fluid through the microcatheter 14. In some embodiments, the infusion system 22 is within the handheld housing 12. In other embodiments, the infusion system 22 is partly or wholly outside the principal housing shell but remains attached to, mounted on, coupled to, or carried by the handheld device assembly so that the device remains handheld during use.

The infusion system 22 may include a fluid chamber, reservoir, syringe, cartridge, bellows, diaphragm chamber, piston chamber, collapsible bag, pressure chamber, tubing path, valve arrangement, pump, injector, manifold, or combinations thereof.

The reservoir and pumping architecture may be selected according to fluid viscosity, required delivered volume, sterility, pressure capability, and user workflow. A syringe or piston chamber may be preferred for predictable volume displacement and compatibility with viscous materials. A cartridge or removable reservoir may be preferred where procedure-specific fluids, rapid replacement, or separation of sterile fluid paths from reusable drive components is desired.

The infused fluid may include viscoelastic, saline, dye, contrast, drug, biologic, therapeutic fluid, diagnostic fluid, irrigating fluid, buffering fluid, implant-release fluid, or combinations thereof.

In some embodiments, fluid is stored within the handpiece. The internal reservoir may be within the principal housing shell or within a housing-mounted cartridge or module. In some embodiments, fluid is stored in an external reservoir that remains part of the handheld assembly. For example, the reservoir may be in a rear-mounted cartridge, side-mounted chamber, clip-on pack, external but hand-borne module, or removable handheld chamber, as generally shown in FIG. 5.

In some embodiments, the device 10 is configured to accept interchangeable reservoirs. One reservoir may contain an ophthalmic viscoelastic device, and another may contain saline, contrast, dye, or a drug. A reusable drive may interface with a disposable sterile reservoir and fluid path.

In some embodiments, an internal reservoir may be preferred because it may simplify setup, reduce the number of separate components presented to the surgeon, reduce priming steps, and permit a more self-contained sterile workflow. In other embodiments, an external but handheld reservoir may be preferred because it may allow larger fluid volume, simpler reservoir replacement, easier visualization of remaining volume, improved modularity between reusable and disposable portions, or reduced size within the principal handpiece shell.

The infusion system 22 may deliver fluid by one or more modes, including pressure-based delivery, pressurized delivery, displacement-based delivery, syringe-type volume exchange, continuous delivery, intermittent delivery, pulsatile delivery, bolus delivery, indexed delivery, metered delivery, rate-controlled delivery, pressure-regulated delivery, pressure-limited delivery, closed-loop delivery, open-loop delivery, or combinations thereof.

Fluid may be delivered under pressure. Pressure may be generated by a pump, motor-driven piston, syringe plunger, diaphragm, bellows, gas pressure source, compressed chamber, pressure cartridge, spring-driven pressure source, or other pressurization arrangement.

Pressurized delivery may facilitate viscodilation, tissue separation, opening of channels, penetration into fine structures, delivery into collector channels, displacement of debris, implant assistance, or treatment of localized resistance. Pressure may be constant, variable, ramped, pulsed, stepped, pressure-limited, feedback-controlled, or selected according to the procedure.

In some embodiments, pressure-based delivery may be preferred because delivery may be controlled based on a pressure target, pressure profile, or pressure response within the system or tissue. In other embodiments, displacement-based delivery, including syringe-volume-exchange delivery, may be preferred where precise metering of small volumes is desired, because a known displacement may correlate with a known delivered volume and may provide predictable dosing. In some embodiments, both pressure-based and displacement-based delivery systems may generate a highly pressurized delivery condition, and either approach may be used to promote penetration into distal outflow anatomy or to induce shear thinning of a shear-sensitive fluid during delivery. This may be used where tissue expansion, viscodilation, distal penetration, or overcoming localized resistance is desired. For example, a displacement-driven system may generate substantial pressure when advancing fluid through a narrow or resistive pathway, and a pressure-based system may likewise be configured to achieve high local shear and elevated delivery pressure. Accordingly, the distinction between pressure-based and displacement-based delivery may relate more to the primary mode of control or regulation than to whether the system is capable of generating pressurized flow.

In some embodiments, fluid delivery is displacement-based. A known displacement of a piston, plunger, diaphragm, bellows, or fluid chamber corresponds to a known infused volume. In some embodiments, the system performs syringe-type volume exchange. Delivery may be linear, incremental, indexed, proportional, or calibrated to account for compliance, temperature, and fluid rheology.

In some embodiments, the infusion system 22 is configured to produce pulsatile flow. Pulsatile flow may be beneficial for penetration into side branches or collector channels, for mobilizing debris, for revealing flow patterns, for accentuating filling defects, for reducing average pressure while maintaining useful pressure peaks, or for better simulating physiologic flow behavior. Pulse amplitude, pulse rate, duty cycle, waveform, and pattern may be fixed or adjustable.

In some embodiments, the infused fluid comprises an ophthalmic viscoelastic device, including hyaluronic acid, sodium hyaluronate, hyaluronan-based materials, chondroitin sulfate-containing materials, hydroxypropyl methylcellulose-containing materials, cohesive materials, dispersive materials, viscoadaptive materials, or combinations thereof.

In some embodiments, the fluid exhibits shear-thinning behavior. Shear-thinning may be advantageous because effective viscosity may decrease under high shear conditions during delivery while remaining relatively higher at lower shear conditions after delivery. This may improve deliverability while preserving beneficial tissue-support or dilation properties.

In some embodiments, shear-thinning materials may be preferred over Newtonian or less shear-responsive materials because they may flow more readily during delivery through restricted pathways while maintaining greater apparent viscosity after exiting the catheter, thereby combining injectability with space-maintaining or tissue-supporting behavior. In some embodiments, less viscous materials may be preferred for mapping or penetration, whereas more viscous materials may be preferred for sustained canal dilation, tissue separation, implant support, or controlled local residence.

In some embodiments, the device 10 includes one or more shear-inducing elements or pathways. Such structures may include narrowed regions, tapered regions, non-uniform pathways, contraction-expansion geometries, ridges, microfeatures, helical paths, textured surfaces, restricted sections, valves, nozzles, or other shear-generating structures. In some embodiments, one or more flow paths are intentionally narrowed or non-uniform to increase shear rate and reduce apparent viscosity of a shear-thinning fluid during infusion.

In some embodiments, a lower-viscosity fluid is used to improve penetration into fine outflow structures such as collector channels, aqueous veins, or small channels. Lower viscosity may reduce flow resistance in accordance with Poiseuille-type flow behavior and other fluid-dynamic relationships, thereby improving penetration into narrow anatomical pathways.

In some embodiments, the system selectively delivers a lower-viscosity fluid for mapping, penetration, or diagnosis and a higher-viscosity fluid for viscodilation, tissue support, implant assistance, or prolonged effect.

The microcatheter 14 may include one or more side ports for fluid delivery. Side ports may be near the distal tip, spaced proximally from the tip, distributed circumferentially, or arranged longitudinally or helically. Side ports may permit lateral infusion into Schlemm's canal, collector channels, surrounding tissue, implant interfaces, or adjacent anatomy.

In some embodiments, the microcatheter 14 includes dual lumens or multiple lumens. A first lumen may carry an ophthalmic viscoelastic device. A second lumen may carry contrast, dye, saline, a drug, or another fluid. The user may select which lumen is active on demand, and the system may switch between lumens through valves, manifolds, selectors, electronic controls, or combinations thereof.

In some embodiments, dual-lumen arrangements support sequential or simultaneous delivery of therapeutic and diagnostic materials. One lumen may be optimized for higher-viscosity materials and another for lower-viscosity materials. One lumen may support infusion while another supports aspiration, sensing, or optical transmission.

In some embodiments, multiple lumens may be preferred over a single-lumen arrangement because they may permit selective on-demand delivery of different materials without withdrawing and exchanging instruments or purging of the line inter-or intra-operatively. For example, one lumen may be optimized for an ophthalmic viscoelastic device and another for contrast or drug delivery, thereby allowing a surgeon to alternate between treatment and diagnostic assessment during a single pass of the microcatheter.

The device 10 may deliver therapeutic or diagnostic materials including ophthalmic viscoelastic devices, saline, balanced salt solutions, dyes, contrast agents, fluorescent agents, radiographic contrast agents, pressure-lowering drugs, anti-inflammatory drugs, anti-fibrotic agents, anti-scarring agents, biologics, implant-release fluids, other diagnostic or therapeutic agents, and combinations thereof.

In some embodiments, the device 10 is used for canal-based angiography, channelography, or analogous outflow mapping procedures. A contrast agent, dye, fluorescent material, optically detectable fluid, or other diagnostic medium may be delivered into Schlemm's canal, collector channels, or related outflow structures.

Such procedures may assist in identifying occlusions, stenoses, partial blockages, herniations, abnormal flow regions, outflow asymmetry, collector channel patency, treatment targets, post-treatment response, implant placement suitability, bypass routes, or regions of elevated resistance.

Channelography or angiography may be performed before treatment, during treatment, after treatment, or multiple times. Pulsatile or pressure-modulated delivery may improve visualization of filling defects or dynamic behavior. Different illumination colors or wavelengths may be used together with contrast delivery to enhance imaging or direct visualization.

In some embodiments, canal-based angiography or channelography may be used not only to identify occlusions or herniations but also to guide treatment strategies by revealing which sectors appear more patent, which collector channels fill preferentially, and where additional viscodilation, drug delivery, implant placement, or repeat catheterization may be beneficial. In some embodiments, pre-treatment and post-treatment imaging may be compared to assessing procedural effects in real time.

The device 10 may be used to identify and/or treat occlusions, stenoses, constrictions, or tissue herniations. Such abnormalities may be inferred from pressure response, infusion resistance, flow asymmetry, stagnant contrast, illumination changes, pulsatile filling behavior, or other sensor-derived or visually derived indicators.

Once identified, such regions may be treated by viscodilation, selective pressure delivery, pulsatile infusion, drug delivery, implant delivery, channel bypass, tissue displacement, or other therapeutic actions.

### Implant Delivery Arrangements

In some embodiments, the device 10 is configured to deliver one or more implants. The implant may be placed in Schlemm's canal, a collector channel, the trabecular meshwork, a suprachoroidal region, a subconjunctival region, or another ocular location.

Implant delivery may be assisted by fluid, including an ophthalmic viscoelastic device. For example, an OVD may lubricate the pathway, expand tissue, maintain space, or help release an implant from the catheter.

In some embodiments, fluid-assisted implant delivery may be preferred because viscoelastic or other delivery fluid may lubricate the implant path, protect tissue during deployment, maintain a target space, reduce friction during release, or provide a hydraulic assist for final positioning. In other embodiments, mechanical release, sheath retraction, spring release, or shape-recovery deployment may be preferred where more discrete positional control or reduced dependence on fluid pressure is desired.

In some embodiments viscoelastic can be the hydraulic fluid which assists in delivery.

The implant may be released by hydraulic force, pressure, mechanical push, sheath retraction, spring release, shape recovery, dissolvable retention, magnetic actuation, thermal actuation, electrical actuation, or other means. The implant may be delivered through a distal opening, a lumen, a side port, a carrier, a sheath, or another release arrangement.

In some embodiments, the implant comprises a filament element which may be positioned fully externally to or partially within an internal passage of the microcatheter prior to deployment within the eye. The filament element may be entirely within the internal passage or may extend partially beyond a distal end of the microcatheter.

In some embodiments, the implant may be made from a metal including nitinol and stainless steel or a polymer including polypropylene, nylon and polyimide. The implant may be a surgical suture which can be loaded into or attached to the device. It can also be pre-loaded into the device and supplied to the surgeon ready to deploy. The suture can be straight or curved or have a shape such as coils or zigzags. The suture can be of various sizes but not limited to 10-0, 9-0, 80, 7-0, 6-0, 5-0, 4-0, 3-0, 2-0, 1-0. The size can be chosen depending on the method of implantation. Smaller sutures such as a 10-0 or 9-0 may be easier to load into the catheter for hydraulic deployment. This may be able to be done without other attachment means required.

The suture can be pre-cut to length and optionally shaped before being partially loaded into the catheter device. Polypropylene for example may show a memory when shaped and as such may be able to be coiled around a former or mandril prior to or during the surgery. This shaping can turn a linear suture into a scaffold. The suture may naturally bend back around the catheter as it is inserted into the eye. It may traverse the canal with the catheter before intentional deployment or may naturally deploy due to the frictional forces within the eye. The catheter may not need to be a catheter with a lumen and may be able to dislodge the suture by gentle advancement and retraction which causes the suture to deploy.

In some embodiments, the microcatheter includes first and second internal elongate members disposed within the internal passage. The first and second internal elongate members may comprise wires, fibres, filaments, strands, optical members, reinforcing members, guide members, sensor carriers, or combinations thereof. In some embodiments, one internal elongate member comprises an optical fiber and another internal elongate member comprises a reinforcing wire or guide member.

In some embodiments, the implant comprising the suture element is releasably retained by engagement between the suture element and the first and second internal elongate members. The engagement may comprise interference engagement, frictional engagement, wedging, pinching, compression, elastic retention, or combinations thereof. In some embodiments, the suture element is lodged between the first and second internal elongate members within the internal passage of the microcatheter.

In some embodiments, the handheld fluid infusion system is configured to deliver a fluid through the microcatheter with sufficient force to dislodge the implant from engagement with the first and second internal elongate members and advance at least a portion of the implant out of the distal end of the microcatheter. In some embodiments, the fluid comprises an ophthalmic viscoelastic material. In some embodiments, the delivered fluid provides a hydraulic assist for implant release and deployment.

In some embodiments, the same fluid used for release also lubricates the implant pathway, expands tissue, maintains space, provides viscodilation, reduces friction during release, or assists final implant positioning. In some embodiments, release occurs without mechanical uncoupling of the implant from a distal external attachment feature.

In some embodiments, the implant is deployed into Schlemm's canal after the microcatheter has been advanced circumferentially or partially circumferentially within the canal. The implant may remain within Schlemm's canal after withdrawal of the microcatheter to provide support, tensioning, dilation, scaffolding, or another therapeutic effect.

In some embodiments the suture may be cut to a length such that the surgeon can facilitate tensioning of the suture which may be done by tying of the two ends together or by attaching a separate joining element such as a crimp segment. The suture may be tied using an ab interno approach within the anterior chamber or even if needed through an ab externo or external scleral approach. The suture may have the ends joined by heat forming or melting or by using a specially designed tool which can grasp both ends and twist or knot them such that they are tied and optionally tensioned.

In some embodiments the implant may not need to be tensioned as the implant itself may provide sufficient benefit by being retained within the eye. The suture shape could provide a scaffold or structure to keep the canal expanded particularly if placed before, during or after dilation of the canal.

In some embodiments Schlemm's canal and the conventional outflow pathway may operate as a dynamic and mechanically responsive system in which tissue motion, pressure variation, pulsatile flow, and local biomechanical signaling contribute to regulation of aqueous humor drainage. In some embodiments, a retained implant positioned within Schlemm's canal may provide therapeutic benefit through one or more mechanisms in addition to dilation. For example, the retained implant may provide a persistent or semi-persistent mechanical interaction with canal tissue, trabecular tissue, collector channel regions, or other adjacent outflow structures, thereby influencing tissue configuration, patency, compliance, motion, responsiveness, or resistance to stenosis. The implant may create a local bias toward openness, maintain separation of tissue surfaces, support restoration or preservation of physiologic motion, alter local strain patterns, modify flow behavior, or otherwise influence the biological and mechanical environment of the canal.

In some embodiments, such interaction may involve stimulation or modulation of mechanosensitive, pressure-responsive, flow-responsive, or otherwise physiologically responsive structures associated with Schlemm's canal and related tissues. As a result, the retained implant may help maintain or promote a more open, functional, and therapeutically favorable outflow state over time, including a state that is less prone to narrowing, collapse, obstruction, or elevated outflow resistance.

In some embodiments, the implant-retention and fluid-release arrangements described herein are used together with any of the handheld illumination, handheld fluid infusion, single-communicating-element, multi-lumen, pressure-controlled, displacement-controlled, channelography, angiography, diagnostic, and therapeutic features otherwise disclosed herein.

### Actuation, Control, Power, Sensors, and Reusable/Disposable Architectures

The fluid infusion system 22 may include a motor or other drive source. The drive source may be an electric motor, a motor driving a piston, syringe, bellows, diaphragm, cam, screw, or pump, a pressurized source, a compressed gas source, a stored-pressure chamber, a wound spring, a compressed spring, an elastic energy store, a piezoelectric actuator, an electromagnetic actuator, a magnetic actuator, an electrostatic actuator, a hydraulic actuator, a pneumatic actuator, a shape-memory actuator, or combinations thereof.

The drive source may be within the housing 12 or outside the principal housing shell while still attached to or borne by the handheld assembly.

Fluid delivery may be actuated by one or more user inputs. In some embodiments, fluid delivery is actuated by an actuator, button, trigger, switch, lever, rocker, thumbwheel press, touch control, or other on-device input.

In some embodiments, fluid delivery is actuated by voice control. The device 10 may include voice recognition hardware or may communicate with a voice-processing subsystem.

In some embodiments, fluid delivery is actuated by eye tracking. The device 10 may include interoperability with vision headsets or eye tracking through a microscope or heads up display. This may allow the surgeon to direct delivery through watching the retraction of the device.

In some embodiments, fluid delivery is actuated by a footswitch or remote-control device. The footswitch may be wired or wireless. Wireless communication may include Bluetooth, infrared, radio frequency, near-field communication, Wi-Fi, optical wireless communication, or other protocols.

The control input may command start, stop, pulse, bolus, aspiration, pressure change, rate change, lumen selection, fluid selection, or synchronization with catheter advancement.

In some embodiments the device may have a prime button or series of buttons which allow the device to be primed either in combination with the user or as an automatic feature. The unit may be able to detect that it has been primed based off measuring the backscatter reflection through a fiber optic or sensor. The device may ramp up or down the priming rate to not over prime the device, causing a loss or waste of fluid eg viscoelastic.

In some embodiments, an on-device button or switch may be preferred because it may allow single-hand control without requiring a separate accessory. In other embodiments, a footswitch may be preferred because it may free the surgeon's hand from infusion control and does not compromise the surgeon's ability to hold the device stable within the eye. Voice control or thought-interface control may be preferred in procedures where reduced manual diversion or hands-busy workflows are desired, provided the control pathway offers suitable reliability and surgical compatibility.

The device 10 may include one or more onboard power sources. The power source 28 may be within the housing 12 or in a module attached to or carried by the handheld device.

Power sources may include lithium-ion batteries, lithium polymer batteries, lithium iron phosphate batteries, primary lithium batteries, silver oxide batteries, alkaline batteries, nickel metal hydride batteries, zinc-air batteries, solid-state batteries, thin-film batteries, microbatteries, flexible batteries, capacitors, supercapacitors, hybrid power systems, removable packs, disposable sterile power modules, rechargeable modules, inductively charged modules, or contact-charged modules.

Power-source selection may depend on current demand, shelf life, sterilization pathway, device reuse model, and allowable handpiece mass. Rechargeable lithium-ion or lithium-polymer cells may be preferred for reusable handpieces with motors, controllers, and illumination. Primary lithium, silver oxide, or alkaline cells may be preferred for single-use or limited-use devices where charging infrastructure is undesirable. Capacitors or supercapacitors may be preferred for short high-power pulses, rapid recharge, or limited-duration illumination or actuation events. Inductive or contact charging may be preferred where sealed reusable housings are desired.

The light source 20 and infusion source may share a power source or have separate power sources.

The device 10 may include one or more controllers, processors, control circuits, memory systems, wireless modules, sensor interfaces, and logic systems. The control system may coordinate catheter advancement, fluid infusion, illumination, pressure, volume, color, pulse timing, and other functions.

The extent of electronic control may be selected according to procedural complexity and desired safeguards. A simple control circuit may be preferred for low-cost devices with limited modes. A processor-based controller may be preferred where catheter advancement, illumination color, infusion pressure, pulse timing, lumen selection, and sensor feedback are coordinated. Memory or communication modules may be useful to store calibration information, identify disposable cartridges, limit reuse, record procedural parameters, or interface with imaging and operating-room systems.

The control system may receive inputs from on-device actuators, voice inputs, footswitches, wireless devices, imaging systems, pressure sensors, displacement sensors, optical sensors, or combinations thereof.

In some embodiments, the device 10 includes sensors configured to detect pressure, flow, displacement, motor current, catheter position, catheter resistance, force, light intensity, optical return signals, tissue interaction, temperature, or other parameters. Sensor information may be used for display, control, safety, diagnostics, or automation.

Sensors may assist in identifying occlusions, entry into target anatomy, resistance changes, proper lumen selection, implant release state, or channel filling patterns.

The type and placement of sensors may be selected according to the parameter most relevant to the intended procedure. Pressure or flow sensing may be preferred for detecting resistance, occlusion, or channel filling. Displacement, motor-current, or force sensing may be preferred for detecting catheter obstruction, excessive friction, or implant release. Optical sensing may be preferred where illumination reflection, fluorescence, optical fringe shift, contrast movement, or catheter visibility is used to infer anatomy or treatment effect. Temperature sensing may be preferred where light output, motor operation, or fluid heating could affect tissue safety.

Pressure sensing at the distal tip may provide direct feedback to sense and prevent damaging delicate structures with excessive pressure during viscodilation. This can reduce potential injury like Descemet's detachments. With an optical method and the use of fiberoptic transmission of light, passive pressure sensing elements can be deployed distally without the need for electrical conductors routed to the distal tip, the location of infusion. This has the advantage of requiring no additional components to clutter the lumen of the distal shaft. An example of such schemes includes the use of fiber Bragg gratings fabricated directly into the structure of the fiber illuminated with broadband light and configured like a spectrum analyzer. With the application of wavelength division multiplexing multiple sensors can be distributed along the fiber length to detect pressure changes in multiple locations simultaneously, acting as a linear array.

Other embodiments include the use of a microfabricated bellows-like cavity located at the distal tip, illuminated and remotely interrogated with coherent light that can be configured as a Fabry-Perot interferometer.

Another embodiment is the use of a hollow optical fiber to transmit light to the distal tip whilst maintaining an internal conduit that can feed relative pressure back to a transducer located in the handpiece, where there is more volume and access to power.

The device 10 may be fully disposable, fully reusable, or a hybrid. In some embodiments, the light source 20 is reusable and the microcatheter 14 is disposable. In some embodiments, the infusion motor is reusable, and the fluid chamber and sterile fluid path are disposable. In some embodiments, the entire device 10 is disposable. In some embodiments, the main handpiece is reusable and accepts disposable catheter-fluidics cartridges, as shown generally.

The disposable, reusable, or hybrid model may be selected according to sterility requirements, cost, environmental considerations, cleaning burden, and performance needs. A fully disposable device may be preferred where sterility assurance and procedural simplicity outweigh component cost. A fully reusable device may be preferred where robust sterilization is feasible and the device incorporates higher-cost drive, optical, or electronic components. A hybrid architecture may provide a practical balance by retaining costly motors, batteries, controllers, and light sources while replacing the catheter, reservoir, sterile fluid path, and patient-contacting components.

Reusable portions may include the housing, motor, controller, battery, communications hardware, voice hardware, light source, and drive mechanism. Disposable portions may include the fluid chamber, sterile tubing, catheter, side-port section, distal delivery assembly, and sterile manifolds.

In one example, a user introduces the handheld device into a surgical field and advances the microcatheter 14 from the housing 12 using the actuator 16 and displacement mechanism 18. The distal portion of the microcatheter 14 is illuminated by the handheld light source 20, either through a dedicated optical element or through the wall, coating, lumen, or fluid column of a single-communicating-element embodiment.

The user then infuses a fluid through the microcatheter 14 using the handheld infusion system 22. The fluid may be an ophthalmic viscoelastic device, saline, contrast, dye, drug, or another material. Delivery may be pressure-based, displacement-based, pulsatile, or otherwise controlled. The user may perform canalography or channelography to identify occlusions or herniations, treat the anatomy with fluid delivery, and optionally deliver an implant.

### Combinations and Definitions

The glaucoma-specific embodiments described below may be combined with any compatible feature in the preceding mechanically aided fluid dispensing embodiments. Conversely, the mechanically aided fluid dispensing embodiments may be adapted for canaloplasty, viscodilation, channelography, angiography, implant delivery, drug delivery, contrast delivery, and other ophthalmic procedures described herein.

The present disclosure is not limited to the specific embodiments described. Features of one embodiment may be combined with features of another embodiment. Elements described as optional may be omitted. Elements described separately may be integrated. Elements described as integrated may be separated. Any disclosed range, material, configuration, delivery mode, actuation mode, illumination mode, or diagnostic mode may be combined with any other compatible disclosed feature unless clearly incompatible.

It will also be appreciated that, unless the context requires otherwise, the terms “comprise”, “comprising”, “include”, “including”, “contain”, “containing”, “have”, “having”, and any variations thereof, used in this document are intended to be understood in an inclusive (i.e. non-exclusive) sense, such that the process, method, device, apparatus, or system described herein is not limited to the features, integers, parts, elements, or steps recited but may include other features, integers, parts, elements, or steps not expressly listed and/or inherent to such process, method, device, apparatus, or system. Further, the terms “a” and “an” used herein are intended to be understood as meaning one or more unless explicitly stated otherwise. Moreover, the terms “first”, “second”, “third”, etc. are used merely as labels, and are not intended to impose numerical requirements on or to establish a certain ranking of importance of their objects. In addition, any reference to positional terms, such as “lower” and “upper”, used in the above description are to be taken in context of the embodiments depicted in the figures, and are not to be taken as limiting the invention to the literal interpretation of the term but rather as would be understood by the skilled addressee in the appropriate context.

The terms used herein are intended to be broad and inclusive. References to ‘a’ or ‘an’ include one or more unless clearly indicated otherwise. References to ‘or’ include any one, any combination, and all listed alternatives unless context requires otherwise. References to ‘handheld’ are intended to encompass structures that remain borne by and manipulated with the instrument even if not physically inside a principal shell of the housing.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

1. An ophthalmic surgical system for glaucoma surgery, comprising:
a handheld surgical device comprising:
a handheld housing;
a microcatheter extendable from the handheld housing; the microcatheter comprising at least one tubular communicating element defining a lumen for fluid delivery and optionally communicating elements configured for optical transmission or sensing;
an actuator disposed on or carried by the handheld housing;
a displacement mechanism operatively coupled to the actuator and configured to advance and retract the microcatheter relative to the handheld housing;
a light source coupled to or contained within the handheld housing, the light source being configured to illuminate at least a distal portion of the microcatheter; and
a fluid infusion system coupled to the handheld housing or carried by the handheld assembly and configured to infuse a fluid through the microcatheter;
wherein each of the light source and the fluid infusion subsystem is independently disposed within the handheld surgical device, carried by the handheld surgical device, or provided in an external unit coupled to the handheld surgical device.

2. The surgical system of claim 1, wherein the device is configured for delivery of an implant comprising a filament.

3. The surgical system of claim 2, wherein the filament is a surgical suture.

4. The surgical system of claim 3, wherein the filament is a surgical suture comprising polypropylene.

5. The surgical system of claim 3, wherein the filament assumes a substantially linear configuration after deployment.

6. The surgical system of claim 3, wherein the filament assumes a non-linear configuration after deployment.

7. The surgical system of claim 2, wherein the delivery is aided by hydraulic fluid.

8. The surgical system of claim 2, wherein the delivery occurs in response to resistance encountered during advancement or withdrawal within Schlemm's canal.

9. The surgical system of claim 2, wherein the implant is long enough to traverse at least 60 degrees of the Schlemm's canal.

10. The surgical system of claim 2, wherein the implant is long enough to traverse at least 360 degrees of the Schlemm's canal.

11. The surgical system of claim 1, wherein the light source is contained within or attached to the device while remaining handheld.

12. The surgical system of claim 1, wherein the microcatheter comprises a first communicating element configured for fluid delivery and a second communicating element configured for optical transmission.

13. The surgical system of claim 1, wherein the at least one tubular communicating element is configured for both optical transmission and fluid delivery.

14. The surgical system of claim 12, wherein the microcatheter comprises at least two lumens configured for fluid delivery.

15. The surgical system of claim 14, wherein the two elements converge within the device system.

16. The surgical system of claim 14, wherein the two elements merge within the device system.
